(19) United States
(12) Patent Application Publication
(10) Pub. No.: US 2026/0219255 A1
(43) Pub. Date: Jul. 30, 2026

(54) Evaluation method of biological nitrogen removal capacity in water based on functional traits of benthic animals

(71) Applicant: Chongqing University, Chongqing (CN)

(72) Inventors: Jingmei Yao, Chongqing (CN); Le Han, Chongqing (CN); Liansen Deng, Chongqing (CN); Yue Zhi, Chongqing (CN); Hongli Li, Chongqing (CN); Qiujun Tan, Chongqing (CN); Chengcheng Wang, Chongqing (CN)

(73) Assignee: Chongqing University, Chongqing (CN)

(21) Appl. No.: 19/451,102

(22) Filed: Jan. 16, 2026

(30) Foreign Application Priority Data
Jan. 24, 2025 (CN) 202510117857.X

(51) Int. Cl. G01N 33/18

(52) U.S. Cl. CPC G01N 33/186; G01N 33/188

(57) ABSTRACT

A method for evaluating nitrogen bio-removal capacity in water based on functional traits of benthic macroinvertebrates, comprising: S1, constructing an evaluation table for the nitrogen bio-removal capacity in water, and S2, collecting the benthic macroinvertebrates in the water to be evaluated, calculating the final functional trait evaluation scores of the benthic macroinvertebrates based on modalities that are strongly correlated with nitrogen bio-removal rate of water indicating the nitrogen bio-removal capacity in water, and then determining corresponding grade based on the evaluation table. The present application requires only the collection and identification of benthic macroinvertebrates from water to obtain the functional trait evaluation result, enabling the determination of the nitrogen bio-removal rate for the water by using the pre-constructed assessment table. The present application significantly reduces the workload, simplifies and accelerates the entire evaluation process, and improves assessment efficiency.

### CROSS-REFERENCE TO RELATED APPLICATIONS AND INCORPORATION BY REFERENCE

The present application claims the benefit and priority of Chinese invention patent application No. 202510117857.X, filed on Jan. 24, 2024, the disclosure of which is incorporated herein by reference in its entirety as part of the present application.

### TECHNICAL FIELD

The present application relates to the field of ecological monitoring and assessment. Specifically, it relates to a method for evaluating nitrogen bio-removal capacity in water based on functional traits of benthic macroinvertebrates, which aims to perform relevant evaluation by utilizing the functional traits of benthic macroinvertebrates.

### BACKGROUND

Nitrogen bio-removal capacity is a critical component of water's self-purification ability. Accurate evaluation of the biological nitrogen removal capacity of streams is of great significance for river health assessment and water environment protection. At present, most of the existing technologies adopt the field in-situ test method based on the nutrient spiraling model to investigate the biological nitrogen removal capacity in water. However, during the implementation of this method, a large number of tracers need to be added to eliminate the impact of abiotic factors in rivers (such as physical advection, dilution, diffusion, etc.) on the biodegradation of nitrogen pollutants. In addition, this method needs to calculate the biological nitrogen removal capacity of streams based on the nutrient spiraling model according to the real-time changes of water quality, making the test and calculation processes rather cumbersome.

In other words, the existing field in-situ test method based on the nutrient spiraling model, which is adopted to investigate the biological nitrogen removal capacity in water, has at least the following difficulties during its implementation: 1) the processes of the test, measurement and calculation are rather cumbersome, 2) it is highly susceptible to interference from abiotic factors in the river, 3) a large amount of tracers need to be used, thus leading to excessively high investigation costs.

Moreover, while the bio-removal of nitrogen in water bodies is primarily mediated by microorganisms and algae, assessment methods focusing on these biological aspects often depend on metrics such as microbial and algal diversity and abundance. A fundamental limitation exists because microorganisms and algae, as primary producers in the aquatic ecosystem, are susceptible to influences from secondary producers. Their efficacy is also readily compromised by abiotic factors (e.g., advection, dilution, diffusion), leading to a poor correlation between these bio-evaluation parameters and the actual nitrogen bio-removal capacity, thereby undermining assessment accuracy.

Consequently, in light of the deficiencies in the prior art summarized above, there is a well-recognized need for a novel method to evaluate nitrogen bio-removal capacity in the water. A superior method would be simpler, more economical, and more feasible than the existing in-situ experimental approaches. It should enable a quick and convenient determination of nitrogen bio-removal capacity in the water while minimizing dependency on sophisticated laboratory instrumentation.

### SUMMARY

The objective of the present application is to provide a method for evaluating nitrogen bio-removal capacity in water based on functional traits of benthic macroinvertebrates. This method addresses the drawbacks of prior art by enabling a convenient, efficient, and precise assessment.

To achieve the above-mentioned objective of the present application, the technical solution adopted is as follows:

The present application provides a method for evaluating nitrogen bio-removal capacity in water based on functional traits of benthic macroinvertebrates, comprising following steps:
S1, constructing an evaluation table for the nitrogen bio-removal capacity in water based on final functional trait evaluation scores of benthic macroinvertebrates, wherein the evaluation table for the nitrogen bio-removal capacity in water is divided into multiple grades, and said grades are classified according to different ranges of the final functional trait evaluation scores of benthic macroinvertebrates,
S2, collecting the benthic macroinvertebrates in the water to be evaluated, calculating the final functional trait evaluation scores of the benthic macroinvertebrates in the water to be evaluated based on modalities that are strongly correlated with nitrogen bio-removal rate of water indicating the nitrogen bio-removal capacity in water, and then determining corresponding grade of the nitrogen bio-removal capacity in the water to be evaluated from the evaluation table pre-constructed for the nitrogen bio-removal capacity in water according to obtained final functional trait evaluation scores of benthic macroinvertebrates.

By implementing the disclosed steps, the present application eliminates the need for the complex tracer addition, real-time water quality monitoring, and cumbersome computational processes characteristic of conventional in-situ field experiments based on the nutrient spiraling model. The method requires only the collection and identification of benthic macroinvertebrates from a stream, followed by correspondence analysis and calculation to derive a final functional trait evaluation score. This score is then referenced against a pre-constructed assessment table to determine the nitrogen bio-removal capacity classification. Compared to the prior art, this method substantially reduces workload, enhances evaluation efficiency, and improves practical feasibility.

Benthic macroinvertebrates are macroscopic aquatic invertebrates inhabiting sediment or attached to aquatic plants or stones. Their sensitivity to water quality makes them effective underwater sentinels. Although they do not directly absorb nutrients, they influence nitrogen bio-removal capacity in the water by exerting up-down control on primary producers through consumption of microorganisms and algae.

Owing to the unique ecological role and position of benthic macroinvertebrates, their functional traits comprehensively reflect environmental adaptations and influences within the food web. An evaluation system constructed on this basis is less susceptible to external interference, thereby overcoming the limitations of methods reliant on microorganisms or algae, which are prone to disturbances from secondary producers and abiotic factors in streams. This results in more stable and reliable assessments with a stronger correlation to the actual nitrogen bio-removal capacity, thus providing a more accurate reflection of real-world conditions and robust support for river health assessment and water environmental protection. In other words, the present application abandons the conventional practice in the prior art of evaluating the biological nitrogen removal capacity in water by means of microorganisms or algae, and adopts the non-obvious approach of using benthic animals to evaluate the biological nitrogen removal capacity in water, thereby overcoming the technical problems inherent in the evaluation methods relying on microorganisms or algae in the prior art.

In the present application, the term “water” refers to rivers, streams, ponds, lakes, and similar aquatic environments. The term “benthic macroinvertebrates” refers to macroscopic, visible aquatic invertebrates that inhabit the bottom substrate or attach to aquatic plants or stones.

Furthermore, the step of constructing an evaluation table for the nitrogen bio-removal capacity in water based on final scores obtained from the evaluation of functional traits of benthic macroinvertebrates comprises:
S11, collecting benthic macroinvertebrate samples at multiple sampling sites, and for each sampling site, constructing a benthic macroinvertebrate abundance matrix based on species and individual counts of collected benthic macroinvertebrates,
S12, constructing a benthic macroinvertebrate functional trait matrix comprising multiple functional traits of benthic macroinvertebrates, based on <Systematic Classification, Biology and Ecology of Freshwater Invertebrates>,
S13, for each sampling site, performing a logarithmic transformation on the constructed benthic macroinvertebrate abundance matrix, and multiplying it by the constructed benthic macroinvertebrate functional trait matrix to obtain a modality value matrix representing the modalities in the functional traits of benthic macroinvertebrates, wherein a parameter for j-th modality of i-th functional trait of benthic macroinvertebrate is denoted as benthic macroinvertebrate modality parameter Aᵢⱼ,
S14, for each sampling site, obtaining nitrogen bio-removal rate of the water based on on-site measurement; and based on the obtained measured nitrogen bio-removal rates, screening out the strongly correlated traits and strongly correlated modalities among the benthic macroinvertebrates that are strongly correlated with nitrogen bio-removal rate of the water,
S15, based on the obtained measured nitrogen bio-removal rates of water, combining with partial least squares regression, calculating and determining first component value for the strongly correlated modalities,
S16, based on the strongly correlated modalities screened, and taking the corresponding first component value of the strongly correlated modalities as modality weight Bᵢ, calculating final functional trait evaluation scores of benthic macroinvertebrates for each sampling site according to the formula X=ΣAᵢⱼ·Bᵢ,
S17, performing a statistical distribution analysis on the final functional trait evaluation scores of benthic macroinvertebrates across all sampling points, and grading the nitrogen bio-removal capacity in the water based on the quantile intervals where the scores fall, so as to obtain the final evaluation table for the nitrogen bio-removal capacity in water.

In the present application, the analysis of a diverse array of functional traits enables a deeper and more refined exploration of the latent correlations between benthic macroinvertebrates and the nitrogen bio-removal capacity in the water. This comprehensive approach mitigates evaluation bias resulting from an incomplete consideration of functional traits. Furthermore, it establishes a solid foundation for the subsequent precise calculation of the functional trait parameter Aᵢⱼ and the accurate assessment of the correlation between nitrogen bio-removal capacity and benthic functional traits. This significantly enhances the scientific rigor and accuracy of the overall evaluation method, yielding more persuasive and reliable results that better serve the research and conservation of stream ecosystems.

Moreover, each step in the present application—from specimen identification and sub-sampling to abundance counting, data transformation, and correspondence analysis—is meticulously designed to ensure the rationality and validity of data processing. This guarantees that the resulting matrices accurately reflect the structure and characteristics of the benthic macroinvertebrate community, thereby providing a reliable data foundation for the subsequent multiplication with the trait matrix to obtain the modality value matrix.

Furthermore, the step of screening out the strongly correlated traits and strongly correlated modalities among the benthic macroinvertebrates that are strongly correlated with nitrogen bio-removal rates of water based on the measured nitrogen bio-removal rates comprises:
taking a logarithm of the measured nitrogen bio-removal rate of water as a dependent variable, and taking the obtained benthic macroinvertebrate modality parameter Aᵢⱼ as predictors, to calculate and obtain the significance value of each modality through a correlation analysis,
selecting the modalities with a significance value of less than 0.05, and identifying functional traits containing modalities with a significance value of less than 0.05 as the strongly correlated functional traits,
performing a regression by taking the logarithm of the measured nitrogen bio-removal rate of water as dependent variable and the obtained benthic macroinvertebrate modality parameters Aᵢⱼ as predictors, and calculating an importance value for each modality within the identified strongly correlated functional traits using variable importance in projection analysis method, and then screening out the modalities with an importance value greater than 0.7, and identifying them as the strongly correlated modalities.

Furthermore, partial least squares (PLS) regression is adopted for regression processing, and the calculation formula of the importance value is as follows:
wherein VIP represents the importance value, p represents initial total number of variables involved in the analysis, h represents final total number of iteration cycles performed, wⱼₖ represents the weight adopted for the mapping of variable j during the k-th iteration, reflecting the degree of interpretation of variable j to the k-th mapping result Xₖ,

$${\hat{c}}_{k}^{2} {t}_{k}^{′} {t}_{k}$$

represents the degree of interpretation of the k-th mapping result Xₖ on Yₖ.

In the present application, the calculation formula for the Variable Importance in Projection (VIP) can accurately quantify the importance of each modality in explaining the biological nitrogen removal capacity in water through complex iterative calculations and weight analysis, so as to screen out the key modalities. Meanwhile, the calculation formula for the final functional trait evaluation score X of benthic animals is based on the screened strongly correlated specific modalities and their corresponding weights, which realizes the quantitative assessment of the biological nitrogen removal capacity in water and endows the evaluation results with clear numerical basis.

Furthermore, the calculation formula of the first component value is as follows:

$$Bi = T {ω}_{1} ;$$

where Bᵢ represents the first component value, T is the modality value matrix, and ω₁ is weight vector obtained through iterative optimization.

Within the computational process of PLS algorithm employed in the present application, the relationship between independent and dependent variables is decomposed and reconstructed. Through data dimensionality reduction and extraction of principal components, the first component value is obtained as a key component value. Its value reflects both the importance and the direction of the influence of the corresponding modality in explaining the nitrogen bio-removal capacity in the water. The sign (positive or negative) of the value indicates the direction of the correlation, with a positive value denoting a positive correlation and a negative value denoting a negative correlation. The absolute magnitude of the value indicates its relative importance within the model, where a larger absolute value corresponds to a greater influence on the nitrogen bio-removal capacity.

Furthermore, a p-value is calculated for each trait within the inventive method. The p-value serves as a key indicator for determining the statistical significance of a trait within the model. If the p-value for a trait is less than a predetermined threshold (typically 0.05), it indicates that the trait possesses statistical significance in explaining the nitrogen bio-removal capacity, meaning a significant linear relationship exists between the trait and the nitrogen bio-removal capacity in the water.

Further, S17 comprises:
based on the calculated final functional trait evaluation scores of benthic macroinvertebrates at all sampling sites, dividing the scores into five quantile intervals as the grades of the nitrogen bio-removal capacity in water, where: if the final functional trait evaluation score of benthic macroinvertebrates is less than the 25th percentile, the nitrogen bio-removal capacity rate of the water is deemed to be low, corresponding to grade V of nitrogen bio-removal capacity in the water,
if the final functional trait evaluation score of benthic macroinvertebrates falls within the range of the 25th to 50th percentile, the nitrogen bio-removal rate of the water is deemed to be relatively low, corresponding to grade IV of nitrogen bio-removal capacity in the water,
if the final functional trait evaluation score of benthic macroinvertebrates falls within the range of the 50th to 75th percentile, the nitrogen bio-removal rate of the water is deemed to be moderate, corresponding to grade III of nitrogen bio-removal capacity in the water,
if the final functional trait evaluation score of benthic macroinvertebrates falls within the range of the 75th to 90th percentile, the nitrogen bio-removal rate of the water is deemed to be relatively high, corresponding to grade II of nitrogen bio-removal capacity in the water,
if the final functional trait evaluation score of benthic macroinvertebrates is greater than the 90th percentile, the nitrogen bio-removal rate of the water is deemed to be high, corresponding to grade I of nitrogen bio-removal capacity in the water,
Furthermore, the multiple functional traits comprise: maximum potential size, life cycle duration, number of cycles per year, aquatic stage, sexual and asexual reproduction, dispersal mode, resistance forms, food, feeding habits, respiration, temperature-suitable microhabitat, locomotion and substrate association.

Furthermore, the strongly correlated functional traits are screened and determined as follows: temperature-suitable microhabitat, locomotion and substrate association, food, and feeding habits.

The strongly correlated modalities are screened and determined as follows:
flagstones/boulders/cobbles/pebbles, silt and mud, microphytes, crawling, interstitial, fine sediment/microorganism, biological detritus, living microphytes, dead animals, and grazing type.

Furthermore, step S2 comprises the following sub-steps:
S21, collecting benthic macroinvertebrates in the water to be evaluated, and constructing a current benthic macroinvertebrate abundance matrix based on species and individual counts of collected benthic macroinvertebrates,
S22, constructing in advance functional trait matrix of macrobenthos in accordance with <Systematic Classification, Biology and Ecology of Freshwater Invertebrates>; performing a logarithmic transformation on the current benthic macroinvertebrate abundance matrix, and multiply it by the pre-constructed benthic macroinvertebrate functional trait matrix to obtain a current modality value matrix representing the modalities in the functional traits of benthic macroinvertebrates, thereby acquiring the benthic macroinvertebrate modality parameter Aᵢⱼ representing the j-th modality of the i-th functional trait in the current modality value matrix,
S23, based on the strongly correlated modalities screened out in step S14, and taking the corresponding first component values of the strongly correlated modalities determined in step S15 as the modality weights Bᵢ, calculating the final functional trait evaluation score of benthic macroinvertebrates for the current test water to be evaluated according to the formula X=ΣAᵢⱼ·Bᵢ,
S24, based on the obtained final functional trait evaluation score of benthic macroinvertebrates for the current water to be evaluated, determining the corresponding grade of the nitrogen bio-removal capacity of the current water to be evaluated from the evaluation table for the nitrogen bio-removal capacity in water constructed in step S1.

Furthermore, collection of benthic macroinvertebrates is carried out by means of a Surber net, and after collection, the benthic animals are placed in a container filled with 95% ethanol for fixation. Specifically, a Surber net is utilized to collect benthic macroinvertebrate samples from the stream. The net is constructed from high-strength, corrosion-resistant nylon material, and its mesh size accuracy is controlled within a tolerance of +5 μm. In the present application, the high-strength, corrosion-resistant nylon construction of the Surber net provides durability and stability in complex stream environments, reducing damage and maintenance costs. The high-precision mesh size control ensures that the collected macroinvertebrate samples are representative and accurate, thereby improving sample quality and providing a reliable data foundation for the entire evaluation method.

The present application applies the evaluation method in the fields of river health assessment and water environment protection. It provides an important technical means for ecological research and conservation, aids in the scientific management of water resources, helps maintain river ecosystem balance, and possesses significant practical importance and social value.

Compared to the prior art, the beneficial effects of the present application are as follows:

The present application, in contrast to traditional in-situ field experimental methods based on the nutrient spiraling model, eliminates the need for complex tracer addition, real-time water quality monitoring, and cumbersome computational processes. The method simply requires collecting and identifying benthic macroinvertebrates from a stream according to the prescribed steps, followed by matrix construction and model calculations, to obtain the evaluation result for the nitrogen bio-removal capacity in the water. This significantly simplifies the evaluation procedure, reduces the workload, improves evaluation efficiency, and makes the entire process more straightforward and practicable. Furthermore, owing to the unique position and role of benthic macroinvertebrates within the ecosystem, their functional traits comprehensively reflect environmental adaptations and influences within the ecological chain. The evaluation system constructed on this basis is less susceptible to external interference, overcoming the problem of methods relying on microorganisms or algae being easily disturbed by secondary producers and abiotic factors in streams. This results in more stable and reliable evaluation outcomes with a stronger correlation to the actual nitrogen bio-removal capacity in the water, enabling a more accurate reflection of the ecological functional status of streams.

The following details disclose the method and system for evaluating nitrogen bio-removal capacity in water bodies based on benthic macroinvertebrate functional traits, as illustrated in the accompanying drawings and marked with reference numerals.

### BRIEF DESCRIPTION OF DRAWINGS

FIG. 1 is a flowchart illustrating the steps of the method for evaluating the nitrogen bio-removal capacity of a water based on benthic macroinvertebrate functional traits according to the present application.

FIG. 2 is a flowchart illustrating the steps of constructing the assessment table for nitrogen bio-removal capacity based on the final functional trait evaluation scores according to the present application.

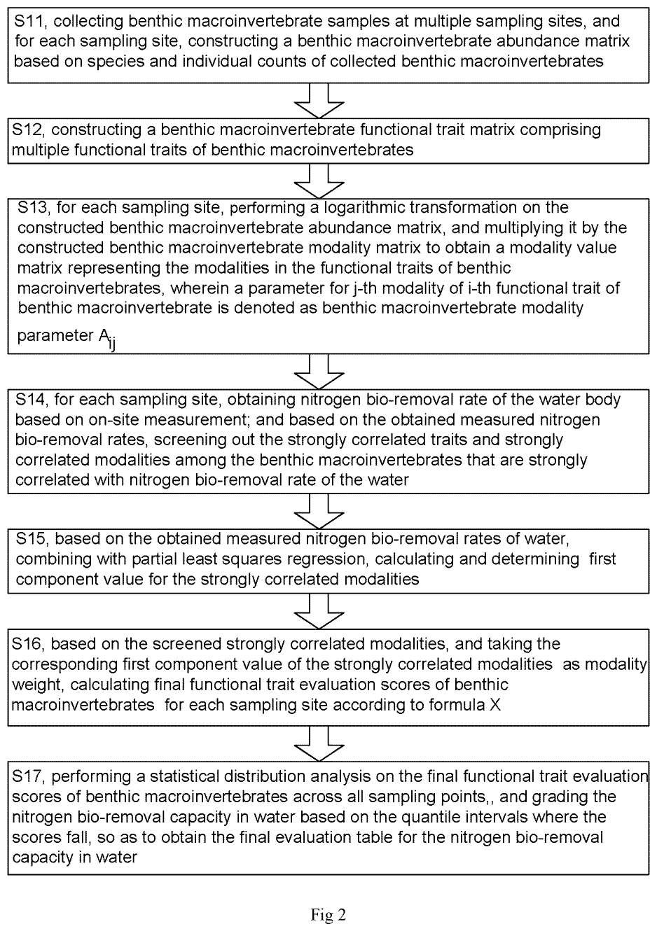

FIG. 3 is a flowchart illustrating the steps of evaluating the nitrogen bio-removal capacity of water using the pre-constructed assessment table according to the present application.

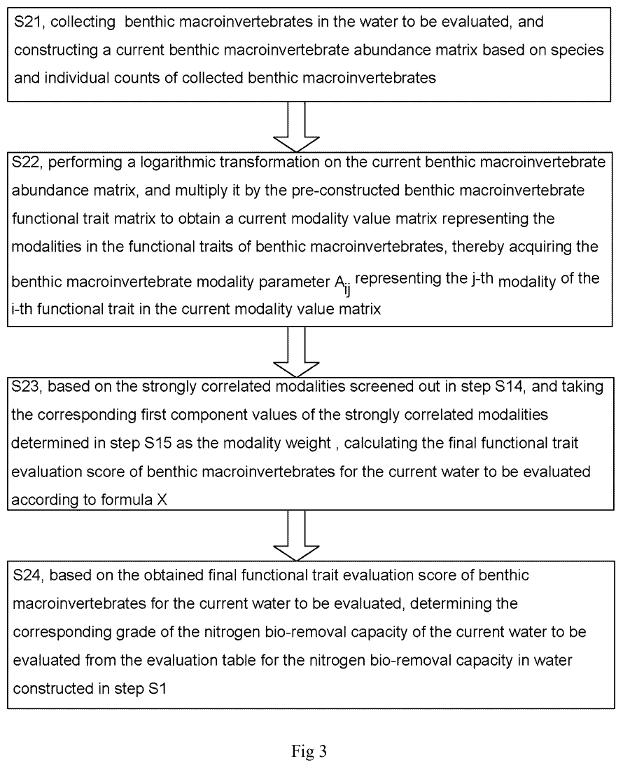

### DETAILED DESCRIPTION

The technical solutions of the embodiments of the present application will be described clearly and completely below with reference to the accompanying drawings. It is to be understood that the embodiments described are merely a part of, but not all, the embodiments of the present application. All other embodiments obtained by a person of ordinary skill in the art based on the embodiments herein without creative efforts shall fall within the scope of the present application. It is noted that the embodiments and features thereof in the present application may be combined with each other if there is no conflict.

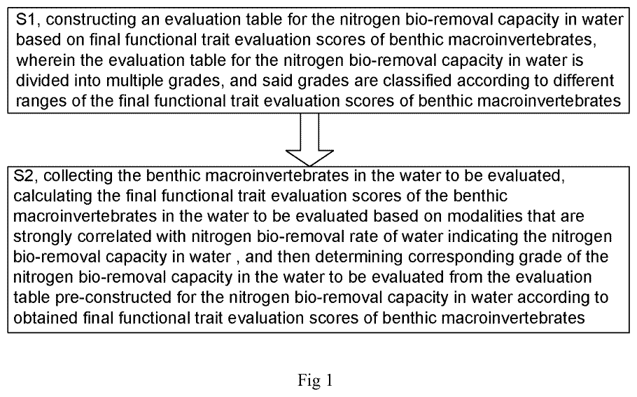

The present application provides a method for evaluating nitrogen bio-removal capacity in water based on functional traits of benthic macroinvertebrates. With reference to FIG. 1, the specific operational steps of the method are as follows:
S1, constructing an evaluation table for the nitrogen bio-removal capacity in water based on final functional trait evaluation scores of benthic macroinvertebrates, wherein the evaluation table for the nitrogen bio-removal capacity in water is divided into multiple grades, and said grades are classified according to different ranges of the final functional trait evaluation scores of benthic macroinvertebrates,
S2, collecting the benthic macroinvertebrates in the water to be evaluated, calculating the final functional trait evaluation scores of the benthic macroinvertebrates in the water to be evaluated based on modalities that are strongly correlated with nitrogen bio-removal rate of water indicating the nitrogen bio-removal capacity in water, and then determining corresponding grade of the nitrogen bio-removal capacity in the water to be evaluated from the evaluation table pre-constructed for the nitrogen bio-removal capacity in water according to obtained final functional trait evaluation scores of benthic macroinvertebrates.

The step of constructing an evaluation table for the nitrogen bio-removal capacity in water based on final scores obtained from the evaluation of functional traits of benthic macroinvertebrates comprises:
S11, collecting benthic macroinvertebrate samples at multiple sampling sites, and for each sampling site, constructing a benthic macroinvertebrate abundance matrix based on species and individual counts of collected benthic macroinvertebrates,
S12, constructing a benthic macroinvertebrate functional trait matrix comprising multiple functional traits of benthic macroinvertebrates, based on <Systematic Classification, Biology and Ecology of Freshwater Invertebrates>,
S13, for each sampling site, performing a logarithmic transformation on the constructed benthic macroinvertebrate abundance matrix, and multiplying it by the constructed benthic macroinvertebrate functional trait matrix to obtain a modality value matrix representing the modalities in the functional traits of benthic macroinvertebrates, wherein a parameter for j-th modality of i-th functional trait of benthic macroinvertebrate is denoted as benthic macroinvertebrate modality parameter Aᵢⱼ,
S14, for each sampling site, obtaining nitrogen bio-removal rate of the water based on on-site measurement; and based on the obtained measured nitrogen bio-removal rates, screening out the strongly correlated traits and strongly correlated modalities among the benthic macroinvertebrates that are strongly correlated with nitrogen bio-removal rate of the water,
S15, based on the obtained measured nitrogen bio-removal rates of water, combining with partial least squares regression, calculating and determining first component value for the strongly correlated modalities,
S16, based on the strongly correlated modalities screened, and taking the corresponding first component value of the strongly correlated modalities as modality weight Bᵢ, calculating final functional trait evaluation scores of benthic macroinvertebrates for each sampling site according to the formula X=ΣAᵢⱼ·Bᵢ,
S17, performing a statistical distribution analysis on the final functional trait evaluation scores of benthic macroinvertebrates across all sampling points, and grading the nitrogen bio-removal capacity in the water based on the quantile intervals where the scores fall, so as to obtain the final evaluation table for the nitrogen bio-removal capacity in water.

In step S11, for sample collection, a Surber net (30 cm×30 cm, 250 μm mesh, constructed from high-strength, corrosion-resistant nylon with a mesh size tolerance controlled within ±5 μm) is used to collect benthic macroinvertebrate samples in the stream. Within a selected stream reach, the Surber net is placed on the substrate surface, ensuring it covers a defined area and rests steadily. The net is pressed into the substrate, and its hinged lid is closed gently to direct benthic organisms into the net. In the present application, the high-strength, corrosion-resistant nylon construction provides durability and stability in complex stream environments, reducing damage and maintenance costs. The high-precision mesh size control ensures the collected macroinvertebrate samples are representative and accurate, thereby improving sample quality and establishing a reliable data foundation for the entire evaluation method.

During collection, take care to minimize excessive disturbance to the stream substrate and surrounding environment to ensure the representativeness of the samples. Multiple collections are performed at different stream locations and depths to obtain sufficiently diverse samples. To demonstrate the feasibility of the invented method, a total of 100 benthic samples were collected from 20 sampling sites according to the analytical method described, ensuring coverage of various microhabitats and biological community compositions. Upon collection, samples are promptly placed in containers with an adequate amount of 95% ethanol for fixation, preventing decomposition and morphological changes, and facilitating subsequent identification and analysis.

In step S11, following sample collection, the process further comprises the step of benthic macroinvertebrate identification and matrix construction. Specifically, constructing a macroinvertebrate abundance matrix includes: first, performing detailed identification and classification on the collected samples to determine the species and count of macroinvertebrates in each sample, utilizing authoritative taxonomic references (e.g., Systematic Classification, Biology, and Ecology of Freshwater Invertebrates) and microscopic examination to achieve the most precise taxonomic level possible. Subsequently, the individual counts for each species are tallied to construct the macroinvertebrate abundance matrix. Following this, a data transformation, typically a logarithmic transformation, is applied to the constructed abundance matrix to meet the mathematical requirements and data distribution characteristics for subsequent analysis.

In step S12, a macroinvertebrate functional trait matrix is constructed based on the aforementioned taxonomic reference. This specifically involves building a matrix encompassing twelve functional traits: maximum potential size, life cycle duration, number of cycles per year, aquatic stages, reproduction, dispersal, resistance forms, food, feeding habits, respiration, substrate (preference), locomotion and substrate association.

In step S13, the constructed macroinvertebrate abundance matrix is multiplied by the functional trait matrix containing the twelve traits, resulting in a modality value matrix T comprising twelve functional traits. The parameter for the j-th state of the i-th functional trait is denoted as Aᵢⱼ. This matrix multiplication is performed using computational software to ensure accuracy and efficiency. During calculation, the dimensions and element values of the matrices are carefully verified to prevent computational errors.

The process proceeds to step S14, namely, performing correlation analysis and trait screening based on measured nitrogen bio-removal rates. This step comprises the following sub-steps:
S1401, the nitrogen bio-removal rate (U_{NO}_{₃}) of a water, which indicates the nitrogen bio-removal capacity, is obtained through actual measurement. Said rate can be acquired by conducting an in-situ field experiment, the implementation process of which can be achieved by prior art methods and will not be elaborated here.
S1402, using the logarithm of the measured nitrogen bio-removal rate as the dependent variable and the obtained benthic modality parameters Aᵢⱼ as predictors, significance value (or referred to as p-value) of each modality is calculated and obtained through correlation analysis,
S1403, modalities with p-value of less than 0.05 are selected, and the corresponding functional traits containing these significant modalities are identified as strongly correlated functional traits,
S1404, performing a regression by taking the logarithm of the measured nitrogen bio-removal rate of water as dependent variable and the obtained benthic macroinvertebrate modality parameters Aᵢⱼ as predictors, and calculating an importance value for each modality within the identified strongly correlated functional traits using variable importance in projection analysis method, and then screening out the modalities with an importance value greater than 0.7, and identifying them as the strongly correlated modalities.

In the correlation calculation of S1402, the logarithm of the nitrogen bio-removal rate is used as the dependent variable, and the obtained benthic macroinvertebrate functional modality parameters Aᵢⱼ are used as predictors. Based on Pearson correlation analysis, a p-value is calculated and acquired for each modality. The formula for calculating the correlation coefficient r is as follows:

$$r = \frac{\sum ( X - \overline{X} ) ( Y - \overline{Y} )}{\sqrt{\sum {( X - \overline{X} )}^{2} {( Y - \overline{Y} )}^{2}}} = \frac{{l}_{XY}}{\sqrt{{l}_{XX} {l}_{YY}}}$$

wherein X is a predictor variable Aᵢⱼ, Y is the logarithm of the nitrogen bio-removal rate. l_{XX} is the sum of squared deviations for X, specifically, l_{XX}=Σ(X−X̅)². l_{YY} is the sum of squared deviations for Y, specifically, l_{YY}=Σ(Y−Y̅)². And l_{XY}=Σ(X−X̅)(Y−Y̅) is the sum of cross-deviations between X and Y.

In the calculation process, a Pearson correlation model function within specialized statistical analysis software (e.g., SPSS, R) may be utilized. The dependent variable and predictor data are inputted, model parameters and analysis options are configured according to the software's operational guidelines, and the p-value for each modality is obtained.

In S1403 for screening functional traits, modalities with a p-value of less than 0.05 are selected based on the results of the Pearson correlation analysis. That is, if a functional trait contains one or more modalities with a p-value <0.05, that functional trait is identified as a strongly correlated functional trait, and all modalities belonging to that functional traits are selected for subsequent testing. Based on the calculated p-values, functional traits meeting the criterion are screened to identify the types of functional traits that have a significant influence on the nitrogen bio-removal capacity in the water. Ultimately, four types of benthic macroinvertebrate functional traits are identified: temperature-suitable microhabitat (i.e., substrate (preference)), locomotion and substrate association, food, feeding habits, with each category containing multiple modalities.

It is specifically noted that the strongly correlated functional traits identified based on the present application, as well as the strongly correlated modalities discussed below, may differ across different aquatic environments and/or different sampling sites within a water. In other words, other combinations of strongly correlated functional traits different from the aforementioned four types in this application are not necessarily excluded from the protectable scope of the present application. The same principle applies to strongly correlated modalities.

To demonstrate the feasibility of evaluating the nitrogen bio-removal capacity of a water using benthic macroinvertebrate functional traits as proposed by the present application, the modality values for the aforementioned four functional traits (temperature-suitable microhabitat, locomotion and substrate association, food, feeding habits) from 20 sample sites are listed in Table 1, according to the analytical method described in the present application.

TABLE 1

| | | Site | | | | | | | | | |
|---|---|---|---|---|---|---|---|---|---|---|---|
| Trait | Modalities | 25 | 26 | 23 | 6 | 24 | 13 | 11 | 8 | 12 | 9 |
| [symbol] temper- | [symbol] | [symbol] | [symbol] | [symbol] | [symbol] | [symbol] | [symbol] | [symbol] | [symbol] | [symbol] | [symbol] |
| ature- | | | | | | | | | | | |
| suitable | | | | | | | | | | | |
| micro- | | | | | | | | | | | |
| habitat | | | | | | | | | | | |
| | [symbol] oulders/ | [symbol] | [symbol] | [symbol] | [symbol] | [symbol] | [symbol] | [symbol] | [symbol] | [symbol] | [symbol] |
| | cobbles/ | | | | | | | | | | |
| | pebbles | | | | | | | | | | |
| | Gravel | 0.11 | 0.11 | 0.11 | 0.11 | 0.12 | 0.11 | 0.11 | 0.11 | 0.11 | 0.11 |
| | Sand | 0.12 | 0.12 | 0.11 | 0.12 | 0.12 | 0.11 | 0.11 | 0.11 | 0.11 | 0.12 |
| | Macrophytes | 0.13 | 0.13 | 0.13 | 0.13 | 0.13 | 0.13 | 0.13 | 0.13 | 0.13 | 0.13 |
| | Microphytes | 0.09 | 0.08 | 0.09 | 0.09 | 0.08 | 0.09 | 0.09 | 0.09 | 0.09 | 0.08 |
| | Organic | 0.11 | 0.11 | 0.11 | 0.11 | 0.11 | 0.11 | 0.11 | 0.11 | 0.11 | 0.11 |
| | detritus/ | | | | | | | | | | |
| | litter | | | | | | | | | | |
| | Silt and mud | 0.22 | 0.22 | 0.22 | 0.23 | 0.21 | 0.22 | 0.22 | 0.22 | 0.22 | 0.21 |
| Locomotion | Crawler | 0.20 | 0.19 | 0.20 | 0.18 | 0.21 | 0.20 | 0.20 | 0.21 | 0.20 | 0.21 |
| and | Burrower | 0.18 | 0.17 | 0.18 | 0.19 | 0.18 | 0.18 | 0.19 | 0.18 | 0.19 | 0.17 |
| substrate | Interstitial | 0.18 | 0.16 | 0.17 | 0.21 | 0.17 | 0.19 | 0.18 | 0.19 | 0.19 | 0.17 |
| association | | | | | | | | | | | |
| Food | Fine | 0.10 | 0.09 | 0.10 | 0.13 | 0.10 | 0.11 | 0.10 | 0.11 | 0.10 | 0.09 |
| | sediments | | | | | | | | | | |
| | and | | | | | | | | | | |
| | microorganisms | | | | | | | | | | |
| [symbol] | [symbol] | [symbol] | [symbol] | [symbol] | [symbol] | [symbol] | [symbol] | [symbol] | [symbol] | [symbol] | [symbol] |
| | | 26 | 26 | 26 | 27 | 27 | 26 | 26 | 27 | 26 | 27 |
| [symbol] | [symbol] | [symbol] | [symbol] | [symbol] | [symbol] | [symbol] | [symbol] | [symbol] | [symbol] | [symbol] | [symbol] |
| | | 14 | 15 | 14 | 16 | 15 | 15 | 15 | 15 | 15 | 15 |
| [symbol] | [symbol] | [symbol] | [symbol] | [symbol] | [symbol] | [symbol] | [symbol] | [symbol] | [symbol] | [symbol] | [symbol] |
| | | 12 | 13 | 12 | 10 | 12 | 12 | 12 | 12 | 12 | 12 |
| [symbol] | [symbol] | [symbol] | [symbol] | [symbol] | [symbol] | [symbol] | [symbol] | [symbol] | [symbol] | [symbol] | [symbol] |
| | [symbol] (=1 mm) | 10 | 09 | 10 | 08 | 10 | 10 | 10 | 10 | 09 | 10 |
| [symbol] | [symbol] | [symbol] | [symbol] | [symbol] | [symbol] | [symbol] | [symbol] | [symbol] | [symbol] | [symbol] | [symbol] |
| | [symbol] brates | 27 | 28 | 27 | 26 | 26 | 27 | 27 | 25 | 27 | 26 |
| | macroin- | | | | | | | | | | |
| | vertebrates | | | | | | | | | | |
| [symbol] | [symbol] | [symbol] | [symbol] | [symbol] | [symbol] | [symbol] | [symbol] | [symbol] | [symbol] | [symbol] | [symbol] |
| | [symbol] feeder | 17 | 16 | 17 | 20 | 17 | 17 | 17 | 18 | 17 | 18 |
| [symbol] | [symbol] | [symbol] | [symbol] | [symbol] | [symbol] | [symbol] | [symbol] | [symbol] | [symbol] | [symbol] | [symbol] |
| | | 16 | 17 | 17 | 16 | 18 | 15 | 16 | 17 | 15 | 17 |
| [symbol] | [symbol] | [symbol] | [symbol] | [symbol] | [symbol] | [symbol] | [symbol] | [symbol] | [symbol] | [symbol] | [symbol] |
| | | 18 | 18 | 18 | 17 | 18 | 18 | 18 | 19 | 18 | 20 |
| [symbol] | [symbol] | [symbol] | [symbol] | [symbol] | [symbol] | [symbol] | [symbol] | [symbol] | [symbol] | [symbol] | [symbol] |
| | | 16 | 15 | 16 | 13 | 15 | 17 | 16 | 15 | 17 | 15 |
| [symbol] | [symbol] | [symbol] | [symbol] | [symbol] | [symbol] | [symbol] | [symbol] | [symbol] | [symbol] | [symbol] | [symbol] |
| [symbol] habits | | 18 | 18 | 18 | 17 | 18 | 18 | 18 | 17 | 18 | 17 |
| | | Site | | | | | | | | | |
| Trait | Modalities | 7 | 17 | 16 | 15 | 2 | 21 | 1 | 18 | 20 | 19 |
| [symbol] temper- | [symbol] | [symbol] | [symbol] | [symbol] | [symbol] | [symbol] | [symbol] | [symbol] | [symbol] | [symbol] | [symbol] |
| ature- | | | | | | | | | | | |
| suitable | | | | | | | | | | | |
| micro- | | | | | | | | | | | |
| habitat | | | | | | | | | | | |
| | [symbol] oulders/ | [symbol] | [symbol] | [symbol] | [symbol] | [symbol] | [symbol] | [symbol] | [symbol] | [symbol] | [symbol] |
| | cobbles/ | | | | | | | | | | |
| | pebbles | | | | | | | | | | |
| | Gravel | 0.11 | 0.11 | 0.11 | 0.11 | 0.11 | 0.12 | 0.11 | 0.11 | 0.12 | 0.12 |
| | Sand | 0.11 | 0.12 | 0.12 | 0.12 | 0.12 | 0.12 | 0.12 | 0.11 | 0.12 | 0.12 |
| | Macrophytes | 0.13 | 0.14 | 0.14 | 0.14 | 0.15 | 0.14 | 0.13 | 0.14 | 0.13 | 0.14 |
| | Microphytes | 0.09 | 0.08 | 0.08 | 0.08 | 0.08 | 0.08 | 0.09 | 0.1 | 0.08 | 0.08 |
| | Organic | 0.11 | 0.11 | 0.11 | 0.10 | 0.11 | 0.11 | 0.11 | 0.11 | 0.11 | 0.11 |
| | detritus/ | | | | | | | | | | |
| | litter | | | | | | | | | | |
| | Silt and mud | 0.22 | 0.22 | 0.22 | 0.22 | 0.22 | 0.20 | 0.23 | 0.20 | 0.20 | 0.19 |
| Locomotion | Crawler | 0.21 | 0.22 | 0.22 | 0.22 | 0.23 | 0.22 | 0.19 | 0.23 | 0.24 | 0.25 |
| and | Burrower | 0.18 | 0.20 | 0.20 | 0.20 | 0.20 | 0.17 | 0.23 | 0.17 | 0.16 | 0.18 |
| substrate | Interstitial | 0.19 | 0.19 | 0.20 | 0.20 | 0.20 | 0.20 | 0.24 | 0.23 | 0.20 | 0.20 |
| association | | | | | | | | | | | |
| Food | Fine | 0.10 | 0.10 | 0.10 | 0.11 | 0.10 | 0.09 | 0.13 | 0.08 | 0.09 | 0.09 |
| | sediments | | | | | | | | | | |
| | and | | | | | | | | | | |
| | microorganisms | | | | | | | | | | |
| [symbol] | [symbol] | [symbol] | [symbol] | [symbol] | [symbol] | [symbol] | [symbol] | [symbol] | [symbol] | [symbol] | [symbol] |
| | | 27 | 27 | 27 | 27 | 29 | 28 | 3 | 29 | 29 | 29 |
| [symbol] | [symbol] | [symbol] | [symbol] | [symbol] | [symbol] | [symbol] | [symbol] | [symbol] | [symbol] | [symbol] | [symbol] |
| | | 15 | 15 | 15 | 16 | 16 | 16 | 17 | 15 | 17 | 16 |
| [symbol] | [symbol] | [symbol] | [symbol] | [symbol] | [symbol] | [symbol] | [symbol] | [symbol] | [symbol] | [symbol] | [symbol] |
| | | 12 | 12 | 11 | 11 | 11 | 12 | 10 | 14 | 13 | 13 |
| [symbol] | [symbol] | [symbol] | [symbol] | [symbol] | [symbol] | [symbol] | [symbol] | [symbol] | [symbol] | [symbol] | [symbol] |
| | [symbol] (=1 mm) | 09 | 09 | 08 | 08 | 08 | 08 | 05 | 09 | 07 | 06 |
| [symbol] | [symbol] | [symbol] | [symbol] | [symbol] | [symbol] | [symbol] | [symbol] | [symbol] | [symbol] | [symbol] | [symbol] |
| | [symbol] brates | 27 | 28 | 28 | 28 | 26 | 26 | 24 | 25 | 25 | 27 |
| | macroin- | | | | | | | | | | |
| | vertebrates | | | | | | | | | | |
| [symbol] | [symbol] | [symbol] | [symbol] | [symbol] | [symbol] | [symbol] | [symbol] | [symbol] | [symbol] | [symbol] | [symbol] |
| | [symbol] feeder | 18 | 17 | 18 | 19 | 19 | 18 | 22 | 16 | 18 | 17 |
| [symbol] | [symbol] | [symbol] | [symbol] | [symbol] | [symbol] | [symbol] | [symbol] | [symbol] | [symbol] | [symbol] | [symbol] |
| | | 17 | 16 | 15 | 15 | 17 | 16 | 16 | 16 | 17 | 18 |
| [symbol] | [symbol] | [symbol] | [symbol] | [symbol] | [symbol] | [symbol] | [symbol] | [symbol] | [symbol] | [symbol] | [symbol] |
| | | 18 | 19 | 18 | 19 | 18 | 20 | 18 | 18 | 20 | 19 |
| [symbol] | [symbol] | [symbol] | [symbol] | [symbol] | [symbol] | [symbol] | [symbol] | [symbol] | [symbol] | [symbol] | [symbol] |
| | | 15 | 16 | 16 | 15 | 14 | 17 | 12 | 18 | 14 | 13 |
| [symbol] | [symbol] | [symbol] | [symbol] | [symbol] | [symbol] | [symbol] | [symbol] | [symbol] | [symbol] | [symbol] | [symbol] |
| [symbol] habits | | 18 | 19 | 19 | 19 | 18 | 17 | 16 | 15 | 16 | 18 |

[symbol]

indicates data missing or illegible when filed

In S1404 for determining the strongly correlated specific modalities, a regression is performed using the logarithm of the nitrogen bio-removal rate as the dependent variable and the obtained benthic macroinvertebrate functional modality parameters Aᵢⱼ as predictors. This regression employs PLS. The VIP value for each functional modality within the selected set of functional indicators is then calculated using VIP analysis. The formula for calculating the VIP value is as follows:

$${VIP}_{j} = \sqrt{\frac{p {\sum }_{k = 1}^{h} ( {\hat{c}}_{k}^{2} {t}_{k}^{′} {t}_{k} ) {( {w}_{jk} )}^{2}}{{\sum }_{k = 1}^{h} {\hat{c}}_{k}^{2} {t}_{k}^{′} {t}_{k}}}$$

wherein p is the initial total number of variables included in the analysis; h is the final total number of iteration cycles performed (yielding h dimensions); wⱼₖ is the weight (i.e., the coefficient in the covariance matrix) assigned to variable j during the k-th iteration (for the k-th dimension), reflecting the contribution of variable j to the k-th component Xₖ.

$${\hat{c}}_{k}^{2} {t}_{k}^{′} {t}_{k}$$

is the contribution of the K-th component Xₖ to explaining the dependent variable Yₖ. The VIP value is analogous to the R² in linear regression, where a higher VIP value indicates a stronger correlation.

The process then proceeds to S15, determining the component 1 value for the strongly correlated specific modalities. In the embodiment of the present application, a total of 10 modalities with VIP>0.7 are screened, and their first component (Component 1) values are determined. A component refers to a latent variable (which can be understood as a value) extracted from the original data. The first component is the initial component extracted during the PLS regression process; it typically captures the majority of the information within the data, explaining a significant portion of the variance in the original data, and thus effectively represents the primary trend in the data. During the calculation, the formula and analytical methods are strictly followed, and statistical software is utilized for computation and analysis to ensure the accuracy of the obtained VIP values and the component 1 values.

Specifically, the first component value Bᵢ is calculated according to the formula:

$$Bi = T {ω}_{1} ;$$

wherein T is the benthic macroinvertebrate functional trait value matrix, and ω₁ is the weight vector obtained through iterative optimization.

The screened ten modalities and their corresponding first component values Bᵢ are presented in Table 2 below.

TABLE 2

| Trait | Modality | Component 1 (Bᵢ) |
|---|---|---|
| | Flagstones/boulders | 0.20 |
| [symbol] | [symbol] | [symbol] |
| | Sand | −0.20 |
| | Microphytes | |
| [symbol] microhabitat | | |
| | Crawler | 0.47 |
| | Interstitial | |
| [symbol] substrate association | | |
| | Fine sediments and | −0.24 |
| | microorganisms | |
| | Detritus | 0.20 |
| | Microphytes | 0.25 |
| | Dead animal (≥1 mm) | |
| | Grazing type | |

[symbol]

indicates data missing or illegible when filed

The process proceeds to the step of calculating the final functional trait evaluation value X for the sampling sites. Specifically, after the final screening of the ten modalities, their corresponding first component values are used as the weights Bᵢ. The final value X is then calculated according to the formula X=ΣAᵢⱼ·Bᵢ, wherein Aᵢⱼ is an element of the benthic functional trait value matrix T, donating the parameter for the j-th state of the i-th functional trait, and Bᵢ is the modality weight.

In S17, the nitrogen bio-removal capacity in the water is classified into five grades based on five quantiles of the calculated final functional trait evaluation value X. The classification is as follows: if X is less than the 25th percentile, the nitrogen bio-removal rate is considered low, corresponding to a Grade V capacity; if X falls between the 25th and 50th percentiles, the rate is considered relatively low, corresponding to a Grade IV capacity; if X falls between the 50th and 75th percentiles, the rate is considered moderate, corresponding to a Grade III capacity; if X falls between the 75th and 90th percentiles, the rate is considered relatively high, corresponding to a Grade II capacity; if X is greater than the 90th percentile, the rate is considered high, corresponding to a Grade I capacity.

By referencing Table 3 below, the nitrogen bio-removal capacity rating of the water can be determined, thereby achieving a graded assessment of the capacity and providing a scientific basis for river health assessment and water environmental protection. Throughout the process, the results of each calculation step are recorded and verified to ensure data reliability and conclusion accuracy.

TABLE 3

| | | Relatively | | Relatively | |
|---|---|---|---|---|---|
| Rate | Low | low | Moderate | high | High |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] trait evaluation | [symbol] | [symbol] | [symbol] | [symbol] | [symbol] |
| score X | | | | | |
| [symbol] | [symbol] | [symbol] | [symbol] | [symbol] | [symbol] |
| [symbol] ) | [symbol] | [symbol] | [symbol] | [symbol] | [symbol] |

[symbol]

indicates data missing or illegible when filed

To demonstrate the feasibility of evaluating the nitrogen bio-removal capacity of a water using benthic macroinvertebrate functional traits as proposed by the present application, and in accordance with the analytical method described herein, the final functional trait evaluation scores (X), the assigned nitrogen bio-removal capacity grades, and the actual measured (U_{NO}_{₃}) values for the 20 sample sites are calculated and listed in Table 4 below.

In this example, the final functional trait evaluation scores X for Sites 25, 26, 23, 6, and 24 are less than 0.1927, indicating a low nitrogen bio-removal rate and corresponding to a Grade V capacity. The scores for Sites 13, 11, 8, 12, 9, and 7 fall within the range of 0.1927 to 0.2070, indicating a relatively low removal rate and corresponding to a Grade IV capacity. The scores for Sites 17, 16, 15, and 2 fall within 0.2070 to 0.2357, indicating a moderate removal rate and corresponding to a Grade III capacity. The scores for Sites 21, 1, and 8 fall within 0.2357 to 0.2492, indicating a relatively high removal rate and corresponding to a Grade II capacity. The scores for Sites 20 and 19 are greater than 0.2492, indicating a high removal rate and corresponding to a Grade I capacity.

As shown in Table 4, the actual (U_{NO}_{₃}) values for the vast majority of sample sites fall within the predicted (U_{NO}_{₃}) range for their respective grades, thereby confirming the favorable predictive performance of the present application.

TABLE 4

| | | | |
|---|---|---|---|
| Sample | The final functional trait | | U_{NO}_{₃}⁻ |
| site | evaluation score X | Grade | (mg*m⁻²*min⁻¹) |

58
59
60
61
62
63
64
65
66
67
68
69
70
71
72
73
74
75
76
77

Once the aforementioned assessment table has been constructed, evaluating the nitrogen bio-removal capacity of a water requires only collecting benthic macroinvertebrates, calculating and obtaining the final functional trait evaluation value, and matching this value against the assessment table to determine the corresponding nitrogen bio-removal capacity classification, thereby achieving the evaluation.

Specifically, the evaluation step S2 for the test water comprises:
S21, collecting benthic macroinvertebrates in the water to be evaluated, and constructing a current benthic macroinvertebrate abundance matrix based on species and individual counts of collected benthic macroinvertebrates,
S22, constructing in advance functional trait matrix of macrobenthos in accordance with <Systematic Classification, Biology and Ecology of Freshwater Invertebrates>; performing a logarithmic transformation on the current benthic macroinvertebrate abundance matrix, and multiply it by the pre-constructed benthic macroinvertebrate functional trait matrix to obtain a current modality value matrix representing the modalities in the functional traits of benthic macroinvertebrates, thereby acquiring the benthic macroinvertebrate modality parameter Aᵢⱼ representing the j-th modality of the i-th functional trait in the current modality value matrix,
S23, based on the strongly correlated modalities screened out in step S14, and taking the corresponding first component values of the strongly correlated modalities determined in step S15 as the modality weights Bᵢ, calculating the final functional trait evaluation score of benthic macroinvertebrates for the current test water to be evaluated according to the formula X=ΣAᵢⱼ·Bᵢ,
S24, based on the obtained final functional trait evaluation score of benthic macroinvertebrates for the current water to be evaluated, determining the corresponding grade of the nitrogen bio-removal capacity of the current water to be evaluated from the evaluation table for the nitrogen bio-removal capacity in water constructed in step S1.

By implementing the disclosed steps, the present application eliminates the need for the complex tracer addition, real-time water quality monitoring, and cumbersome computational processes required by conventional in-situ field experiments based on the nutrient spiraling model. The method requires only the collection and identification of benthic macroinvertebrates from a water, followed by correspondence analysis and calculation to derive a final functional trait evaluation value. This value is then compared against a pre-constructed assessment table to obtain the evaluation result for the nitrogen bio-removal capacity in the water. Compared to the prior art, the method of the present application significantly reduces the workload, improves evaluation efficiency, streamlines the entire process, and enhances practical feasibility.

Furthermore, owing to the unique ecological role and niche of benthic macroinvertebrates, their functional traits comprehensively reflect environmental adaptations and influences within the ecological chain. The evaluation system constructed on this basis is less susceptible to external interference, thereby overcoming the technical problem inherent in methods relying on microorganisms or algae, which are easily disturbed by secondary producers and abiotic factors in streams. This results in more stable and reliable evaluation outcomes with a stronger correlation to the actual nitrogen bio-removal capacity, thus providing a more accurate reflection of real-world conditions and robust support for water health assessment and water environmental protection. In other words, the present application departs from the conventional approach in the prior art of evaluating nitrogen bio-removal capacity using microorganisms or algae. Instead, it adopts the non-obvious solution of employing benthic macroinvertebrates for evaluation, thereby overcoming the technical limitations associated with microorganism- or algae-based methods.

In the present application, the analysis of a diverse array of functional traits enables a deeper and more refined exploration of the latent correlations between benthic macroinvertebrates and the nitrogen bio-removal capacity in the water, thereby mitigating evaluation bias resulting from an incomplete consideration of functional traits. Furthermore, it establishes a solid foundation for the subsequent precise calculation of the benthic functional trait parameter Aᵢⱼ and the accurate assessment of the correlation between nitrogen bio-removal capacity and benthic functional traits. This significantly enhances the scientific rigor and accuracy of the overall evaluation method, yielding more persuasive and reliable results that better serve the research and conservation of stream ecosystems.

Moreover, each step in the present application—from identification and sub-sampling to abundance counting, data transformation, and correspondence analysis—is meticulously designed to ensure the rationality and validity of data processing. This guarantees that the resulting matrices accurately reflect the structure and characteristics of the benthic macroinvertebrate community, thereby providing a reliable data foundation for the subsequent multiplication with the trait matrix to obtain the trait state value matrix.

Furthermore, within the computational process of the PLS algorithm employed in the present application, the relationship between independent and dependent variables is decomposed and reconstructed. Through data dimensionality reduction and extraction of principal components, the component 1 value is obtained as a key component value. Its value reflects both the importance and the direction of the influence of the corresponding trait state in explaining the nitrogen bio-removal capacity. The sign (positive or negative) indicates the direction of the correlation, with a positive value denoting a positive correlation and a negative value denoting a negative correlation. The absolute magnitude indicates its relative importance within the model, where a larger absolute value corresponds to a greater influence on the nitrogen bio-removal capacity.

Furthermore, the present application encompasses twelve functional traits covering multiple aspects, enabling a comprehensive and integrated reflection of the characteristics of benthic macroinvertebrates in stream ecosystems and their relationship with the environment. This provides rich information for accurately assessing their impact on the nitrogen bio-removal capacity in the water, significantly enhancing the accuracy and reliability of the evaluation, thereby avoiding one-sidedness in the assessment.

Moreover, in the present application, the regression algorithm screens the functional traits included in the model by iteratively introducing or removing variables to identify the optimal model fit. During this process, a Pearson correlation analysis is performed to calculate a p-value for each modality. The p-value serves as an important indicator for determining the statistical significance of a modality within the model. If the p-value of a modality is less than a set threshold (typically 0.05), it indicates that the modality has statistical significance in explaining the nitrogen bio-removal capacity, meaning a significant linear relationship exists between the modality and the nitrogen bio-removal capacity in the water. Specifically, setting the p-value threshold at 0.05 and selecting modalities with p-values less than 0.05 effectively excludes those modalities that have minimal or non-significant effects on the dependent variable (nitrogen bio-removal capacity). This helps simplify the model structure, preventing the model from becoming complex and difficult to interpret due to the inclusion of excessive irrelevant or minor variables. The simplified model requires less time and fewer resources for computation and analysis, thereby improving research efficiency. Concurrently, reducing the number of variables helps mitigate issues such as multicollinearity, further enhancing the model's stability and accuracy, and ultimately leading to a more efficient and precise evaluation of the nitrogen bio-removal capacity.

In the present application, the formula for calculating the VIP value enables, through complex iterative computations and weight analysis, the precise quantification of the relative importance of each modality in explaining the nitrogen bio-removal capacity in the water, thereby screening out key modalities. Concurrently, the formula for calculating the final functional trait evaluation value X, based on the screened important modalities and their respective weights, achieves a quantitative assessment of the nitrogen bio-removal capacity, providing the evaluation results with a definitive numerical basis.

Furthermore, in the present application, a VIP threshold is pre-set at 0.7. This threshold is selected to ensure the model retains explanatory power while preventing excessive complexity. If the VIP threshold were set too low, an excessive number of variables might be incorporated, potentially leading to model overfitting—where the model performs well on training data but exhibits poor generalization capability on new data. Conversely, setting the threshold too high might omit certain variables that are important albeit having a relatively weaker influence. Thus, the setting of the VIP threshold at 0.7 establishes a balance, allowing the model to accurately capture the primary influencing factors without becoming unwieldy or difficult to interpret and apply due to an overabundance of variables. This contributes to enhanced model stability and reliability, providing robust support for the accurate assessment of the nitrogen bio-removal capacity.

The present application establishes a link between the functional traits of benthic macroinvertebrates and the nitrogen bio-removal capacity in water, thereby providing a novel methodology and perspective for investigating the biological driving mechanisms of nitrogen cycling in water ecosystems. In contrast to prior art that predominantly focused on the influence of physical and chemical factors on nitrogen removal, the present application emphasizes the importance of biological factors, particularly benthic macroinvertebrates. This contributes to a more comprehensive and in-depth understanding of the material cycling processes and ecological functions within stream ecosystems. It is capable of revealing the role and contribution of different benthic macroinvertebrate functional traits in nitrogen bio-removal, providing ecologists with a specific case study and data support for further research on the relationship between biodiversity and ecosystem functioning, thereby enriching the theoretical framework of ecosystem ecology.

Furthermore, the steps from sample collection to the final determination of capacity are intricately connected, establishing a complete and logically coherent evaluation system for assessing the nitrogen bio-removal capacity in water. This system eliminates the dependency of traditional methods on complex experimental conditions and biological indicators that are prone to interference, thereby providing a new scientific pathway for evaluation and enhancing its systematic nature and accuracy.

The advantages of the method provided in the present application are summarized as follows:

1) It enables the direct determination of the nitrogen bio-removal capacity in the water based on benthic macroinvertebrates, offering convenience and rapidity; 2) It does not impose high requirements for sophisticated instrumentation, demonstrating excellent practicality and potential for broad application; 3) It is cost-effective, as the consumables required are essentially limited to containers and ethanol for field sampling, and the necessary hardware comprises primarily a Surber net and a microscope; 4) It is more economical compared to methods relying on microbial indicators for assessing water nitrogen bio-removal capacity; and 5) Most importantly, it is simpler, more economical, and exhibits higher feasibility than traditional methods employing in-situ field experiments.

Finally, it is to be understood that the aforementioned embodiments are provided merely to illustrate the technical solutions of the present application without limiting its scope. Although the present application has been described in detail with reference to preferred embodiments, a person of ordinary skill in the art will appreciate that modifications or equivalent replacements to the technical solutions of the present application may be made without departing from the spirit and scope of the present application, all of which are intended to fall within the scope defined by the appended claims.

What is claimed is:

1. A method for evaluating nitrogen bio-removal capacity in water based on functional traits of benthic macroinvertebrates, wherein the method comprises following steps:
S1, constructing an evaluation table for the nitrogen bio-removal capacity in water based on final functional trait evaluation scores of benthic macroinvertebrates, wherein the evaluation table for the nitrogen bio-removal capacity in water is divided into multiple grades, and said grades are classified according to different ranges of the final functional trait evaluation scores of benthic macroinvertebrates,
S2, collecting the benthic macroinvertebrates in the water to be evaluated, calculating the final functional trait evaluation scores of the benthic macroinvertebrates in the water to be evaluated based on modalities that are strongly correlated with nitrogen bio-removal rate of water indicating the nitrogen bio-removal capacity in water, and then determining corresponding grade of the nitrogen bio-removal capacity in the water to be evaluated from the evaluation table pre-constructed for the nitrogen bio-removal capacity in water according to obtained final functional trait evaluation scores of benthic macroinvertebrates, the step of constructing an evaluation table for the nitrogen bio-removal capacity in water based on final scores obtained from the evaluation of functional traits of benthic macroinvertebrates comprises:
S11, collecting benthic macroinvertebrate samples at multiple sampling sites, and for each sampling site, constructing a benthic macroinvertebrate abundance matrix based on species and individual counts of collected benthic macroinvertebrates,
S12, constructing a benthic macroinvertebrate functional trait matrix comprising multiple functional traits of benthic macroinvertebrates,
S13, for each sampling site, performing a logarithmic transformation on the constructed benthic macroinvertebrate abundance matrix, and multiplying it by the constructed benthic macroinvertebrate functional trait matrix to obtain a modality value matrix representing the modalities in the functional traits of benthic macroinvertebrates, wherein a parameter for j-th modality of i-th functional trait of benthic macroinvertebrate is denoted as benthic macroinvertebrate modality parameter Aᵢⱼ,
S14, for each sampling site, obtaining nitrogen bio-removal rate of the water based on on-site measurement; and based on the obtained measured nitrogen bio-removal rates, screening out the strongly correlated traits and strongly correlated modalities among the benthic macroinvertebrates that are strongly correlated with nitrogen bio-removal rate of the water,
S15, based on the obtained measured nitrogen bio-removal rates of water, combining with partial least squares regression, calculating and determining first component value for the strongly correlated modalities,
S16, based on the screened strongly correlated modalities, and taking the corresponding first component value of the strongly correlated modalities as modality weight Bᵢ, calculating final functional trait evaluation scores of benthic macroinvertebrates for each sampling site according to formula X=ΣAᵢⱼ·Bᵢ,
S17, performing a statistical distribution analysis on the final functional trait evaluation scores of benthic macroinvertebrates across all sampling points, and grading the nitrogen bio-removal capacity in the water based on the quantile intervals where the scores fall, so as to obtain the final evaluation table for the nitrogen bio-removal capacity in water.

2. The method of claim 1, wherein the step of screening out the strongly correlated traits and strongly correlated modalities among the benthic macroinvertebrates that are strongly correlated with nitrogen bio-removal rate of the water based on the obtained measured nitrogen bio-removal rates comprises:
taking a logarithm of the measured nitrogen bio-removal rate of water as a dependent variable, and taking the obtained benthic macroinvertebrate modality parameter Aᵢⱼ as predictors, to calculate and obtain the significance value of each modality through a correlation analysis,
selecting the modalities with a significance value of less than 0.05, and identifying functional traits containing modalities with a significance value of less than 0.05 as the strongly correlated functional traits,
performing a regression by taking the logarithm of the measured nitrogen bio-removal rate of water as dependent variable and the obtained benthic macroinvertebrate modality parameters Aᵢⱼ as predictors, and calculating an importance value for each modality within the identified strongly correlated functional traits using variable importance in projection analysis method, and then screening out the modalities with an importance value greater than 0.7, and identifying them as the strongly correlated modalities.

3. The method according to claim 2, wherein step S17 comprises:
based on the calculated final functional trait evaluation scores of benthic macroinvertebrates at all sampling sites, dividing the scores into five quantile intervals as the grades of the nitrogen bio-removal capacity in water, where:
if the final functional trait evaluation score of benthic macroinvertebrates is less than the 25th percentile, the nitrogen bio-removal rate of the water is deemed to be low, corresponding to grade V of nitrogen bio-removal capacity in the water if the final functional trait evaluation score of benthic macroinvertebrates falls within the range of the 25th to 50th percentile, the nitrogen bio-removal rate of the water is deemed to be relatively low, corresponding to grade IV of nitrogen bio-removal capacity in the water,
if the final functional trait evaluation score of benthic macroinvertebrates falls within the range of the 50th to 75th percentile, the nitrogen bio-removal rate of the water is deemed to be moderate, corresponding to grade III of nitrogen bio-removal capacity in the water, if the final functional trait evaluation score of benthic macroinvertebrates falls within the range of the 75th to 90th percentile, the nitrogen bio-removal rate of the water is deemed to be relatively high, corresponding to grade II of nitrogen bio-removal capacity in the water,
if the final functional trait evaluation score of benthic macroinvertebrates is greater than the 90th percentile, the nitrogen bio-removal rate of the water is deemed to be high, corresponding to grade I of nitrogen bio-removal capacity in the water.

4. The method according to claim 3, wherein partial least squares regression is adopted for regression processing, and the calculation formula of the importance value is as follows:
$${VIP}_{j} = \sqrt{\frac{p {\sum }_{k = 1}^{h} ( {\hat{c}}_{k}^{2} {t}_{k}^{′} {t}_{k} ) {( {w}_{jk} )}^{2}}{{\sum }_{k = 1}^{h} {\hat{c}}_{k}^{2} {t}_{k}^{′} {t}_{k}}}$$
wherein VIP represents the importance value, p represents initial total number of variables involved in the analysis, h is final total number of iteration cycles performed, wⱼₖ represents the weight adopted for the mapping of variable j during the k-th iteration, reflecting the degree of interpretation of variable j to the k-th mapping result Xₖ,
$${\hat{c}}_{k}^{2} {t}_{k}^{′} {t}_{k}$$
represents the degree of interpretation of the k-th mapping result Xₖ on Yₖ.

5. The method according to claim 4, wherein the calculation formula of the first component value is as follows:
$$Bi = T {ω}_{1}$$
where Bᵢ represents the first component value, T is the modality value matrix, and ω₁ is weight vector obtained through iterative optimization.

6. The method according to claim 5, wherein the multiple functional traits comprise: maximum potential size, life cycle duration, number of cycles per year, aquatic stage, sexual and asexual reproduction, dispersal mode, resistance forms, food, feeding habits, respiration, temperature-suitable microhabitat, locomotion and substrate association.

7. The method according to claim 6, wherein the strongly correlated functional traits are screened and determined as follows: temperature-suitable microhabitat, locomotion and substrate association, food, and feeding habits,
the strongly correlated modalities are screened and determined as follows:
flagstones/boulders/cobbles/pebbles, silt and mud, microphytes, crawling, interstitial, fine sediment/microorganism, biological detritus, living microphytes, dead animals, and grazing type.

8. The method according to claim 1, wherein step S2 comprises:
S21, collecting benthic macroinvertebrates in the water to be evaluated, and constructing a current benthic macroinvertebrate abundance matrix based on species and individual counts of collected benthic macroinvertebrates,
S22, performing a logarithmic transformation on the current benthic macroinvertebrate abundance matrix, and multiply it by the pre-constructed benthic macroinvertebrate functional trait matrix to obtain a current modality value matrix representing the modalities in the functional traits of benthic macroinvertebrates, thereby acquiring the benthic macroinvertebrate modality parameter Aᵢⱼ representing the j-th modality of the i-th functional trait in the current modality value matrix,
S23, based on the strongly correlated modalities screened out in step S14, and taking the corresponding first component values of the strongly correlated modalities determined in step S15 as the modality weight Bᵢ, calculating the final functional trait evaluation score of benthic macroinvertebrates for the current water to be evaluated according to the formula X=ΣAᵢⱼ·Bᵢ,
S24, based on the obtained final functional trait evaluation score of benthic macroinvertebrates for the current water to be evaluated, determining the corresponding grade of the nitrogen bio-removal capacity of the current water to be evaluated from the evaluation table for the nitrogen bio-removal capacity in water constructed in step S1.

9. The method according to claim 8, wherein collection of the benthic macroinvertebrates is carried out by means of a Surber net, and after collection, the benthic animals are placed in a container filled with 95% ethanol for fixation.
